(19) United States
(12) Patent Application Publication
(10) Pub. No.: US 2012/0078147 A1
(43) Pub. Date: Mar. 29, 2012

(54) COMPRESSION GARMENTS PROVIDING TARGETED AND SIMULTANEOUS COMPRESSIVE THERMAL THERAPY

(75) Inventors: Amy G. Ogulnick, Port Washington, NY (US); Andrew Nipon, New York, NY (US); Steve Petitt, Atlantic Beach, FL (US)

(73) Assignee: Recover Gear, LLC, Ponte Verde, FL (US)

(21) Appl. No.: 13/183,423

(22) Filed: Jul. 14, 2011

Related U.S. Application Data
(63) Continuation-in-part of application No. 11/744,257, filed on May 4, 2007.
(60) Provisional application No. 61/364,268, filed on Jul. 14, 2010.
(60) Provisional application No. 60/746,487, filed on May 4, 2006.

(51) Int. Cl. A61F 5/37

(52) U.S. Cl. 602/2

(57) ABSTRACT

A therapeutic compression garment having an the inner layer of compression fabric and an outer layer of gradient compression fabric that also includes an area of zoned compression, wherein a pocket is defined between the outer and inner layers of fabric, is positioned under the area of zoned compression, and is accessible through a slit formed in the outer layer of fabric, wherein the pocket is strategically positioned within the garment overlying a respective body part of the wearer of the garment, and wherein the pocket receives an insertable thermal medium therein, whereby the insertable thermal medium is held securely in place and applies compressive thermal therapy to the respective underlying body part of the wearer as a function of the compression applied by the inner and outer layers of fabric.

### CROSS REFERENCE TO RELATED APPLICATIONS

This application claims the priority benefit of U.S. Provisional Patent Application Appl. No. 61/364,268, filed Jul. 14, 2010, and is a continuation-in-part (CIP) of pending U.S. Nonprovisional patent application Ser. No. 11/744,257, filed May 4, 2007, which claimed priority benefit of now expired U.S. Provisional Patent Appl. No. 60/746,487, filed May 4, 2006. The present application incorporates herein by reference all of the above-referenced applications, as if each were set forth herein in its entirety.

### FIELD OF THE PRESENT INVENTION

The present invention relates generally to therapeutic compression garments, and, more particularly, to high performance compression garments providing zoned and gradient compression and having a dual layer of compressive seamless fabric that allows pockets to be defined therebetween at selective locations, accessible through slits or similar openings in the outer layer of the compressive fabric, for easily receiving and securely holding in place thermal media, such as ice or heat packs, that allows the application of targeted, compressive, and uniform thermal therapy to desired body part locations of the wearer of such a garment.

### BACKGROUND OF THE PRESENT INVENTION

### A. Thermal Therapy

Thermal therapy conventionally involves the application of heat or cold to tissue in an effort to heal and rehabilitate injuries such as bruises, sprains, or other trauma to bone, muscle, ligaments, tendons, and tissue and to treat degenerative conditions and inflammatory diseases and disorders. In a cold application, a cold medium is applied to an affected area to diminish swelling and inflammation, to reduce pain, and to promote healing of the injured body part(s). In a heat application, a hot medium is applied to an affected area to help loosen extremity tissue and joint tissue, such as muscles, ligaments and tendons. Application of heat promotes stretching and increases the flow of blood and oxygen to the affected tissue or other body part. The application of heat serves to increase the range of motion and improve the flexibility in an individual's muscle, ligaments, tendons, and tissue; thus, improving the functionality, comfort and performance of the targeted body parts or anatomical areas of the individual. Heat is typically applied prior to an athletic endeavor or several days after injury to a specific body part. In contrast, cold is most effective when applied to an injured or physically taxed body part immediately after strenuous activity using that body part or injury to that body part.

The medical and sports industries are continually searching for improved methods to expedite recovery and healing from injury or trauma to the body—regardless of the cause of such injury or trauma. One recovery method which has become the choice (and bane) of many athletes is an ice bath, which is a form of cold thermal therapy. After intense exercise or strenuous muscular activity, individuals typically have microscopic tears in their muscles that cause inflammation. Similarly, strained or pulled muscles, ligaments, or tendons also tend to swell or become inflamed after an injury. The chilling temperature of an ice bath acts as a vasoconstrictor, causing blood vessels to contract and drain blood from chilled areas. After leaving the bath, fresh blood flows into the previously contracted vessels, invigorating the muscles, ligaments, and tendons with oxygen and, in turn, reducing inflammation. This increase in blood flow also makes its way deep into the muscle tissue, helping flush out any build-up of lactic acid. The combined effect of supplying oxygen to muscles while flushing out lactic acid facilitates recovery of muscles, tendons, bones, nerves, and other body tissue.

Unfortunately, there are many disadvantages to ice baths. For example, immersing large portions of one's body into an ice bath causes intense discomfort and severe pain Another disadvantage is that an ice bath indiscriminately chills the entire submerged portions of the body—even if only a selected subportion of that body part needs the cold therapy. Therefore, not only are the affected muscle groups exposed to the cold temperatures, but all neighboring parts of the anatomy must endure the intense cold of an ice bath, even if no benefit is attainable for those neighboring body parts. Additionally, ice baths are most effective when taken within about 60 minutes of finishing rigorous activity or after an acute injury. However, ice baths are not portable and are often inaccessible immediately after an athletic activity or injury. Further, ice baths do not typically allow for active recovery, which is the application of cold therapy while simultaneously allowing the individual to be mobile, which, when combined, increases blood flow, reduce stiffness, and has been shown to reduce the overall effects of trauma or injury to a body part and to help speed up recovery.

Concomitantly, other conventional thermal therapies do not facilitate targeting a plurality of muscles, ligaments, tendons, and tissues simultaneously with a thermal medium. Instead, such other conventional thermal therapies typically entail wrapping one targeted area at a time. The wraps may be difficult to apply, and frequently shift after they are applied. Consequently, an individual making use of an ice pack or two is usually only able to target one, or at best, two body parts that need to be “iced down.” Using ace bandages or tape are somewhat effective for holding one or two ice packs, but they require time and effort to put on. Having the individual hold the ice pack on the affected area (if the area can even be reached) is also another alternative, but obviously has its drawbacks and limitations. Typically, there are no easy ways to simultaneously and easily target multiple injury or trauma sites with thermal therapy, especially if all of the areas need to be treated with cold therapy within that critical 60 minute window after the trauma or injury occurs.

### B. Compression Garments

In a related but separate vein, sports scientists are constantly looking for new ways to take human athletic performance to the next level. Compression garments represent a way of safely and legally manipulating human physiology to produce an internal environment that is more conducive to high performance and faster recovery. The science behind compression garments is not new and compression garments have been used medically for many years to treat venous insufficiency, edema and prevent deep vein thrombosis in post operative patients. However, the positive effects of using compression garments to improve or enhance athletic is performance has only recently been discovered and continues to be studied.

For example, recent research in trained athletes reported that compression garments increased VO2max by 10% and anaerobic threshold by 40%. Given that these two physiological variables are highly correlated to success in endurance sports, compression garments appear to offer a significant competitive advantage for endurance athletes.

Additionally, it has been suggested that excess oscillatory displacement of a muscle during a dynamic movement may contribute to fatigue and interfere with neurotransmission and optimal muscle recruitment patterns. Compression garments have been shown to reduce, significantly, longitudinal and anterior-posterior muscle oscillation during specific athletic maneuvers and to increase the efficiency and power of muscles that have been wrapped or enclosed within a compression garment.

Yet further, it is well known that high intensity exercise produces lactic acid, which, in turn, causes muscle fatigue and impaired athletic performance. Studies have shown that compression garments decrease blood lactate concentrations 15 minutes following high intensity exercise when compression garments were worn during and after exercise, which improves athletic performance during exercise, reduces muscle fatigue, and promote quicker recover.

### C. Combined Compression Garments and Compressive Thermal Therapy

Applicant has previously described and taught the benefits of combining simultaneous wearing of compressive garments and applying compressive thermal therapy to targeted body parts using strategically placed pockets on the outside of such high performance, athletic compressive garments, particularly when the pockets help compress the thermal packs on the targeted body parts. Such garments solve several of the problems and issues described above. However, there is still a need for improved compression garments that provide zoned and gradient compression and that enable thermal therapy to be applied in numerous locations or on numerous body parts simultaneously and in a manner that enhances the healing and recovery process. preferably include dual layers of compressive fabric that define pockets therebetween, accessible through slits or similar openings in the outer layer of the compressive fabric, for receiving and tightly holding in place thermal media, such as ice or heat packs, that provide the dual benefits of use of compression garments as well as simultaneous application of compressive thermal therapy to targeted body parts or anatomical areas of the wearer of one of the garments—all of which enhance the wearer's athletic performance and enable faster recover to multiple muscle groups, joints, tendons, and ligaments because targeted compressive heat therapy can be applied to numerous body part locations during warm-ups and during actual exercise, and then targeted cold compressive therapy can be applied quickly and easily, immediately after exercise while wearing the same garment, to the same or different body parts of the wearer of the garment. The improved pocket design combined with the zoned, gradient, and transitional compression garment fabric design provides for an improved compression garment and one that more easily allows compressive thermal therapy to be applied uniformly to a wider range of muscle groups and other body parts, to expanded coverage of such muscle groups and other body parts—all of which are easily and quickly selectable by the wearer of the garment.

### SUMMARY OF THE PRESENT INVENTION

To overcome one or more of the issues described above, Applicant has designed an improved compression garments that provide zoned and gradient compression and that enables thermal therapy to be applied in numerous locations or on numerous body parts simultaneously and in a manner that enhances the healing and recovery process. Preferably, such compression garments providing zoned and gradient compression and include a dual layer of compressive seamless fabric that allows pockets to be defined therebetween at selective locations, accessible through slits or similar openings in the outer layer of the compressive fabric, for easily receiving and securely holding in place thermal media, such as ice or heat packs, that allows the application of targeted, compressive, and uniform thermal therapy to desired body part locations of the wearer of such a garment. Such garments can be used to apply targeted, compressive heat therapy to numerous body part locations during warm-ups, rehabilitation or physical therapy sessions, during exercise or actual sporting events. Similarly, these same garments can be used to apply targeted cold compressive therapy quickly and easily, immediately after exercise or shortly after an injury or other body trauma. The improved pocket design, which provides large pocket spaces between the dual compression garment layers, combined with the actual compression garments in which the fabric layers provide zoned, gradient, and transitional compression features, provides for an improved compression garment and one that more easily allows compressive thermal therapy to be applied uniformly to a wider range of muscle groups and other body parts, to expanded coverage of such muscle groups and other body parts—all of which are easily and quickly selectable by the wearer of the garment.

In a preferred embodiment, improved therapeutic compression garments provide improved compression and thermal therapy benefits not heretofore available—particularly for individuals in the immediate period of time just after injury, trauma, or strenuous athletic activity. In addition, improved pocket designs and pocket locations used with such compression garments have been shown and described herein that provide for more exact and enhanced thermal therapy at strategic muscle/joint/tendon/ligament locations and with improved and uniform coverage of desired muscle, joint, ligament, and tendon groups that are likely to need and benefit from immediate thermal therapy for warm-ups and stretching before athletic or rehabilitative activities or immediately after an injury, trauma, exercise, or rehab.

Preferably, the garments provide and are intended to provide or deliver gradient compression to all body parts underlying such garments and to apply additional, targeted or zoned compression to targeted body parts areas. The specific level of compression will vary by garment and by the intended use of such garment. The particular fabrics and weaves used to create zoned, compression garments are known to those skilled in the art. Further, it is possible and often desirable to include transition areas in which the level of compression gradually decrease as one moves away from a targeted or zoned compression area to a standard gradient compression area of the garment.

In another preferred embodiment, the design of the compression garments described herein takes advantage of the seamless technology offered by circular knitting machines to provide a garment that offers maximum comfort and flexibility, particularly when being used for warm-ups or during physical activity, while also providing maximum, uniform coverage and efficacy of compressive thermal therapy to desired areas of the wearer's body. Circular or seamless fabric construction does this by creating an equal surface pressure to keep either hot or cold pack (or sheets, as they may sometimes be called) not only in place but also under compression, and in a manner in which the thermal medium conforms to the contour or physique of the underlying body part being treated. Preferably, pockets for holding the thermal media are created and defined between dual fabric layers of the compression garment.

Preferably, the dual layers of fabric used to create such compression garments are made using a circular, multi-function knitting machine. Each of these garments is anatomically designed to create graduated or gradient compression—with highest compression further from the heart and lowest compression closer to the heart. Additionally, the compression is preferably zoned, meaning that support and compression provided by the garment increases and decreases based on its proximity to large muscle groups that are typically targeted for thermal therapy. This is preferably accomplished through the selection and use of certain weaves within the fabric. The balanced surface pressure that compression garments provide triggers improved blood circulation, which delivers more oxygen to working muscles. Better circulation also enables the body to eliminate lactic acid and other metabolic wastes that can cause muscle fatigue. The result is an anatomically specific graduated compression garment that fits like a second skin. Yet further, through the use of transitional areas between zoned compression areas that provide the highest level of compression and non-zoned areas that provide the least level of compression, the garments described herein provide significant flexibility and range of movement and minimize any restriction to the wearer's movement while wearing such compression garments—whether or not thermal therapy is being applied at the time.

In a preferred embodiment, the fabric used to create the compression garments described herein are made from high performance, 4-way stretch yarns. This fabric design improves comfort, mobility, and moisture control in both the inner and outer layer, when the compression garment includes dual layers. Additionally, such fabric allows for an efficient transfer of the thermal therapy to the desired body part of the wearer. In some embodiments, the thickness of the two layers of the garment are substantially the same. In other embodiments, the inner layer of the garment (i.e., the one closest to the skin of the wearer is thinner than the outer layer of fabric to improve the thermal transfer between the wearer's body and any thermal packs inserted in pockets between the dual layers of fabric.

In one embodiment, both layers of the garment have gradient and zoned compression properties. In other embodiments, the inner layer of the garment is substantially, uniformally compressive, but the outer layer of the garment provides the gradient and zoned and transitional compressive aspects of the garment as a whole.

The pocket architecture of the improved compression garment described herein are preferably achieved through the ability of the circular knitting and 4 way stretch fabric to reduce the number of seams needed to create what is referred to as the pocket. Preferably, such pockets are formed and created between the dual layers of compression fabric used to create the compression garment. The outer layer of the garment provides the necessary zoned and gradient compression, which is sufficient to hold ice or heat packs (or similar thermal therapy media) in place and in compression, while still allowing for maximum mobility and comfort for the wearer of the compression garment. Although the fabric used to make the compression garments is described as circular or seamless fabric construction, the compression garments described herein use seams or hems to attached the two layers of the garment together at desired or strategic locations. Such seams or hems are used to create and reinforce the pocket openings, are used to attached the two layers of the fabric together to define the outer confines of different pockets contained between the two layers of garment fabric, and to provide other reinforcement horizontally or vertically along the document. Preferably, such seams or hems are designed and use threads that enable the seam or hem to stretch with the surrounding garment fabric and without inhibiting the elastic, flexibility, stretchability and recoverability of the overall compression garment. Using the space between the dual layers of compression garment fabric to define and place the pockets for holding the thermal media enables pocket openings to be placed in a wide range of locations, depending upon the type of garment involved and the particular uses for which that garment will be used. Such pocket design also allows for the pockets to be larger than traditional pockets, whether they be sewn behind the primary or only layer of fabric used in conventional garments or whether they are attached to the outside surface of a traditional garment. Thus, the present compression garments provide for maximum and targeted coverage for use of thermal therapy. In other words, thermal therapy is not limited to small pocket areas, but to almost any space, and, likewise, to any underlying body part of the wearer that needs application of thermal therapy.

Specific compression garments include, but are not limited to: shorts, which are preferably intended to cover the quads, hamstrings, it band, hip flexors, and lower lumbar region; knickers, which are similar to shorts but also preferably include coverage of the patella and surrounding tendons; tights, which are similar to knickers but also preferably include coverage of the shin and calf; long pants, which are similar to tights but which go down to or over the ankles; shin and calf sleeves, which are preferably intended to cover the shins and calves, respectively; knee sleeves, which are preferably intended to cover the front, radial, and rear portions of the knee; elbow sleeves; wrist sleeves; wrist/hand gloves or glove/sleeve combinations; forearm sleeves, full arm sleeves, which are preferably intended to cover the wrist, forearm, and elbow (front and rear); short sleeve shirts, which are preferably intended to cover the shoulder head (front and rear), scalpula, L1 and L2, L4, and L5 of the vertebrae; long sleeve shirts, which are preferably intended to cover everything that the short sleeve shirt covers plus includes the same coverage provided by the arm sleeves, plus coverage of the biceps and triceps muscle groups; long socks and/or ankle sleeves, which are preferably intended to cover the ankle (inside and out), the top of the foot, and in some embodiments the areas affected by plantar fasciitis; full body tights, which are intended to cover the same areas as long or short sleeve shirts in combination with one of shorts, knickers, tights, and long pants. Compression garments may also include girdles or similar body wraps that are designed only to cover some or all of the torso part of the body, but none of the appendages as well as any type of head garments, such as a ski mask with or without coverage of any facial areas.

The above features as well as additional features and aspects of the present invention are disclosed herein and will become apparent from the following description of preferred embodiments of the present invention.

### BRIEF DESCRIPTION OF THE DRAWINGS

Further features and benefits of the present invention will be apparent from a detailed description of preferred embodiments thereof taken in conjunction with the following drawings, wherein similar elements are referred to with similar reference numbers, and wherein:

FIG. 1 illustrates a front plan view of an exemplary short-sleeve shirt having shoulder pockets according to principles of a first embodiment of the invention;

FIG. 2 illustrates a back plan view of the exemplary short-sleeve shirt in FIG. 1 further showing a lumbar pocket according to principles of the first embodiment of the invention;

FIG. 3 illustrates a front plan view of exemplary shorts having quadriceps pockets according to principles of the first embodiment of the invention;

FIG. 4 illustrates a back plan view of the exemplary shorts in FIG. 3 further showing hamstring muscle group pockets according to principles of the first embodiment of the invention;

FIG. 5 illustrates a front plan view of exemplary pants having quadriceps pockets according to principles of the first embodiment of the invention;

FIG. 6 illustrates a back plan view of the exemplary pants in FIG. 3 further showing hamstring and calf muscle group pockets according to principles of the first embodiment of the invention;

FIG. 7 illustrates a front plan view of exemplary pants having stirrups and quadriceps and ankle pockets according to principles of the first embodiment of the invention;

FIG. 8 illustrates an exemplary pocket and thermal media insert for use with any compression garment according to principles of the first embodiment of the invention;

FIG. 9 illustrates a back plan view of exemplary pants having stirrups and hamstring, calf and ankle pockets according to principles of the first embodiment of the invention;

FIG. 10 illustrates a front plan view of an exemplary shirt having elbow and shoulder pockets according to principles of the first embodiment of the invention;

FIG. 11 illustrates a back plan view of an exemplary shirt having elbow and shoulder pockets according to principles of the first embodiment of the invention;

FIG. 12 illustrates a side plan view of exemplary socks having ankle pockets according to principles of the first embodiment of the invention;

FIG. 13 illustrates a front plan view of an exemplary carrying case for thermal media and garments according to principles of the first embodiment of the invention;

FIG. 14 illustrates a front plan view of an exemplary sleeve having a plurality of pockets according to principles of the first embodiment of the invention;

FIGS. 15a-15d illustrate front and back plan views of exemplary shorts, illustrating gradient and zoned compression areas, use of dual fabric layers, and pocket opening locations according to principles of a second embodiment of the invention;

FIGS. 16a-16d illustrate front and back plan views of an exemplary long sleeve shirt, illustrating gradient and zoned compression areas, use of dual fabric layers, and pocket opening locations according to principles of the second embodiment of the invention;

FIGS. 17a-17d illustrate front and back plan views of an exemplary long sock, illustrating gradient and zoned compression areas according to principles of the second embodiment of the invention; and

FIGS. 18a-18d illustrate front and back plan views of an exemplary sock sleeve, illustrating gradient and zoned compression areas of a sock sleeve that is designed to be worn over a sock according to principles of the second embodiment of the invention.

Those skilled in the art will appreciate that the invention is not limited to the exemplary embodiments depicted in the figures or the shapes, relative sizes, proportions or materials shown in the figures.

### DETAILED DESCRIPTION OF PREFERRED EMBODIMENTS

The garments described herein are designed for use by men and/or women, adults and/or children, and may be used as an outergarment or an undergarment. As used herein, pants broadly refer to a garment extending from about the wearer's waist to the legs. Pants may extend to the ankles, calves, knee, below the knee or above the knee. As used herein, shirt broadly refers to a garment that covers all or part of the wearer's upper torso and possibly the wearer's arms and neck. Shirts may include long sleeves, short sleeves, no sleeves, turtle necks, no necks, and may extend below, to or above the wearer's waist. Shorts refer to a garment worn over the pelvic area and the upper part of the upper legs or more, possibly extending as far as mid-calf, but not covering the entire length of the leg.

In one preferred embodiment, garments according to principles of the invention are made close fitting and, preferably, are compression garments. In a particular preferred embodiment, the garments are comprised of an elastic material and configured to conform to the wearer's body. As used herein, “elastic material” refers to a material that is capable of being easily expanded and resuming former shape. Something that has the ability to resume its former shape after expansion is also referred to herein as being “recoverable.” Preferably, the compression garments conform to the general area of the body to be supported without excessive stretching that would cause discomfort to the wearer or damage the material. However, the garment may need to be stretched in order to tightly fit and conform to the general area of the body to be treated.

Illustratively, a shape recovery fabric that can be placed in tension and upon release return to its original shape may be utilized. The shape recovery fabric may be comprised of elastic fibers, or a blend of substantially elastic and other fibers. The elastic fibers may comprise spandex or elastane synthetic fibers. Spun from a block copolymer, spandex fibers exploit the high crystallinity and hardness of polyurethane segments, yet remain “rubbery” due to alternating segments of polyethylene glycol. This enables stretching repetitively without breaking and still recovering the original length. The material is also lightweight; abrasion resistant; soft; smooth; supple; resistant to body oils, perspiration, lotions, and detergents. By way of example and not limitation, FIREWALL™ fabric by Sugoi Performance Products; DuPont Corporation's LYCRA™, and CLEERSPAN™ by the Globe Manufacturing Co. are suitable elastic materials that may be used. The elastic fibers may be blended with cotton, polyester, nylon and/or other suitable fibers alone, with other fibers or in various combinations to provide an elastic material suitable for a compression garment according to principles of a first embodiment of the invention.

In a preferred construction, the garment material may be woven to provide equal four-way stretch capabilities, meaning that it stretches equally along all four planar axes. Alternatively, the woven elastic material may be formed so that it stretches more in one direction than another direction, such as allowing the material to stretch more or less in a horizontal direction than in a vertical direction.

As used herein, the term pocket broadly refers to any compartment formed on the garment that is suitable for holding a thermal medium tightly or in compression against a part of the body underlying such pocket. FIG. 8 provides a close-up plan view of an exemplary pocket 800 according to the first embodiment of the invention. The exemplary pocket 800 is comprised of a panel 805 of pocket material attached to the garment along edges, except at least one edge. The unattached edge 810 defines an opening to a compartment between the attached panel 805 and the main fabric layer of the garment, such as pants, shorts, a shirt or socks. The pocket material may be comprised of the same material as the garment or a different material compatible with the garment. The pocket material may also include moisture impermeable and/or insulating materials and/or layers to protect thermal media within the pocket. Releasable closures, such as hook and loop fasteners, zippers, drawstrings, buttons and/or snaps, may optionally be provided along the unattached edge to seal the pocket in a closed configuration, thereby securing any contents within the compartment. However, the pocket is preferably designed using compressive fabric such that the compression provided by the pocket fabric itself is sufficient to hold the thermal media in place within the pocket and compressively against the underlying body part of the wearer of the garment.

Pockets are preferably sized and positioned to target determined anatomical areas (muscles, tendons, bones, nerves and other tissue) of the wearer's body. By way of illustration and not limitation, for an adult's garment, a quadriceps pockets 715, 750 may be approximately 8″W×10″L and located on the front part of the middle upper thigh. Major muscle groups targeted by quadriceps pockets 715, 750 include, for example, Sartorius, Rectus Femoris, Vastus Medialis, and Vastus Lateralis. As discussed above, closures may be provided at the open edge of each of the pockets to releasably close the pocket and thereby secure its contents.

The pockets are adapted to hold thermal media 815 for applying compressive thermal therapy to the underlying targeted areas. Various thermal media now known in the pertinent art and hereafter developed may be utilized for delivering thermal therapy to a targeted area. Such media may include (without limitation) ice and heat packs and pads and hot water bottles. The principles of the invention apply to both cold thermal media and hot thermal media, both of which are intended to come within the scope hereof. Preferably, flexible or malleable media are used to enable the media to conform closely to the wearer's physique and physical contours. In a particular preferred embodiment, Techni Ice™ reusable dry ice packs/gel packs sold by Techni Ice Australia Pty Ltd are utilized for hot and cold therapies.

Those skilled in the art will appreciate that unlike mere conventional garments having pockets, high performance, athletic compression garments according to the present invention feature a tight form fitting design, with pockets strategically positioned over key muscle groups, joints, tendons, and ligaments, and combinations of the above. In addition, the pockets are sized to receive thermal media and cover all or a substantial portion of the underlying muscle groups, joints, tendons, and ligaments, and combinations of the above. Furthermore, the tight form fitting compression garment design of the fabric, including that used for the pocket, maintains the thermal media firmly against the underlying muscle groups, joints, tendons, and ligaments, and combinations of the above being treated to enhance the therapeutic effect. Concomitantly, the high performance, athletic compression garment also provides overall support and compression to targeted muscle groups, even if thermal therapy is not being applied, which, in and of itself, enhances the recovery process for the wearer of the garment.

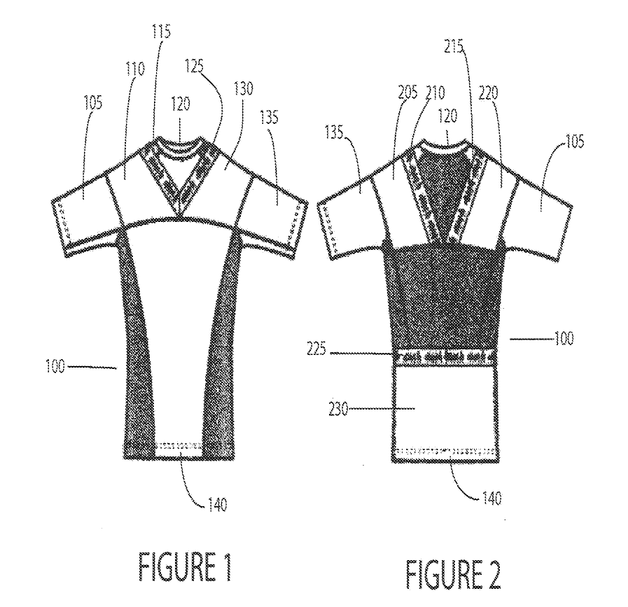

With reference to the drawings, wherein like numerals represent like features, a first embodiment of several exemplary high performance, compression garments are illustrated and described in more specific detail with reference to FIGS. 1-14. Front and rear plan views of an exemplary short-sleeve shirt 100 having shoulder pockets 110, 130 and a lumbar pocket 230 according to principles of the first embodiment of the present invention are shown in FIGS. 1 and 2, respectively. The shirt 100 may be comprised of an elastic, compressive garment material or fabric as described above. An open waist with a waistband 140, a pair of upper arm coverings 105, 135 an arm opening in each arm covering 105, 135 and a neck opening 120 are provided. The shirt 100 is preferably made to fit tightly over and conform to the contours or physique of the body of the wearer of the garment. Various panels of material may be stitched together in conventional manner to form the shirt. While the exemplary arm coverings 105, 135 extend to about the wearer's biceps, shorter (or longer) arm coverings may optionally be used.

Each pocket 110, 130 includes an opening with an elastic band 110, 130 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

Referring now to FIG. 2, a rear view of the exemplary shirt 100 from FIG. 1 having rear shoulder pockets 205, 220 according to principles of the first embodiment of the present invention are shown. Additionally, a lumbar pocket 230 is provided. Each pocket 205, 220, 230, includes an opening with an elastic band 210, 215, 225 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

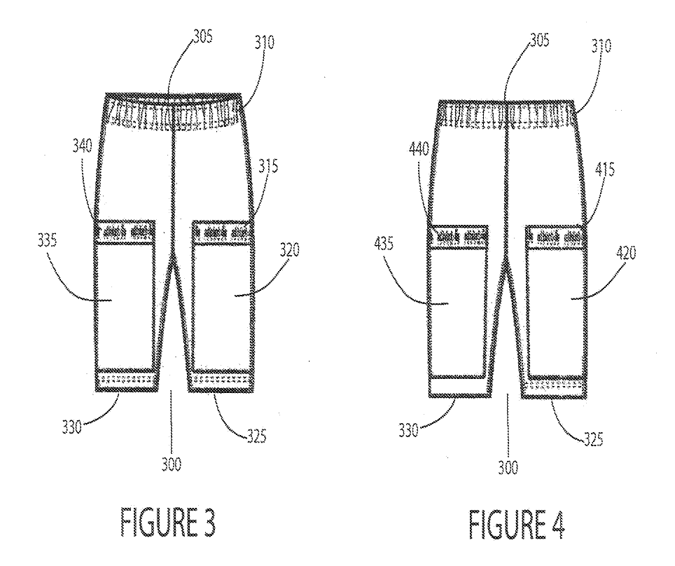

Referring now to FIG. 3, a front view of an exemplary stretchable, form-fitting shorts 300 having right and left quadriceps pockets 320, 335 according to principles of the first embodiment of the invention is shown. The pants may be comprised of an elastic, compressive garment material or fabric, as described above. An open waist 305 with a waistband 310, a pair of short leg coverings 325, 330 with a leg opening in each leg covering are provided. The shorts 300 are preferably made to fit tightly over and conform to the contours or physique of the body of the wearer of the garment. Various panels of material may be stitched together in conventional manner to form the pants. While the exemplary leg coverings 325, 330 extend to about the wearer's thighs, shorter (or longer) leg coverings may optionally be used.

Each pocket 320, 335 includes an opening with an elastic band 315, 340 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

Referring now to FIG. 4, a rear view of the exemplary shorts 300 from FIG. 3 having hamstring pockets 420, 435 according to principles of the first embodiment of the invention is shown. Each pocket 420, 435 includes an opening with an elastic band 415, 440 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

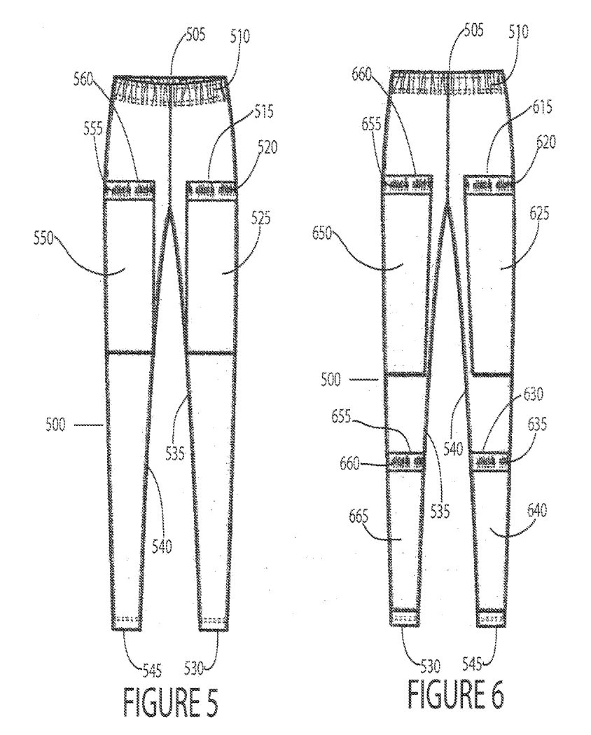

Referring now to FIG. 5, a front view of exemplary stretchable, form-fitting pants 500 having right and left quadriceps pockets 525, 550 according to principles of the first embodiment of the invention is shown. The pants may be comprised of an elastic material as described above. An open waist 505 with a waistband 510, a pair of leg coverings 535, 540 and an ankle opening 530, 545 in each leg covering are provided. The pants 500 are preferably made to fit tightly over and conform to the contours or physique of the body of the wearer of the garment. Various panels of material may be stitched together in conventional manner to form the pants. While the exemplary leg coverings 535, 540 extend to about the wearer's ankles, shorter (or longer) leg coverings may optionally be used.

Each pocket 525, 550 includes an opening 515, 560 with an elastic band 520, 555 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

Referring now to FIG. 6, a rear view of the exemplary pants 500 from FIG. 5 having hamstring pockets 625, 650 according to principles of the first embodiment of the invention is shown. Additionally, calf pockets 640, 665 are provided.

Each pocket 625, 640, 650, 665 includes an opening 615, 630, 655, 660 with an elastic band 620, 635, 655, 660 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

Referring now to FIG. 7, a front view of exemplary stretchable, form-fitting pants 700 having right and left quadriceps pockets 710, 750 according to principles of the first embodiment of the invention is shown. The pants may be comprised of an elastic material as described above. An open waist with a waistband 705, a pair of leg coverings 720, 745 and an ankle opening in each leg covering are provided. The pants 700 are preferably made to fit tightly over and conform to the contours or physique of the body of the wearer of the garment. Various panels of material may be stitched together in conventional manner to form the pants. While the exemplary leg coverings 720, 745 extend to about the wearer's ankles, shorter (or longer) leg coverings may optionally be used. Optionally, stirrups 730, 735 may be provided to ensure that the bottom of the leg coverings remain in place at or near the ankles when the pants are worn. Additionally, optional ankle pockets 725, 740, may be provided along either side, both sides, or the back and/or front of the ankle area of the pants 700.

Each pocket 715,750 includes an opening with an elastic band 710, 755 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

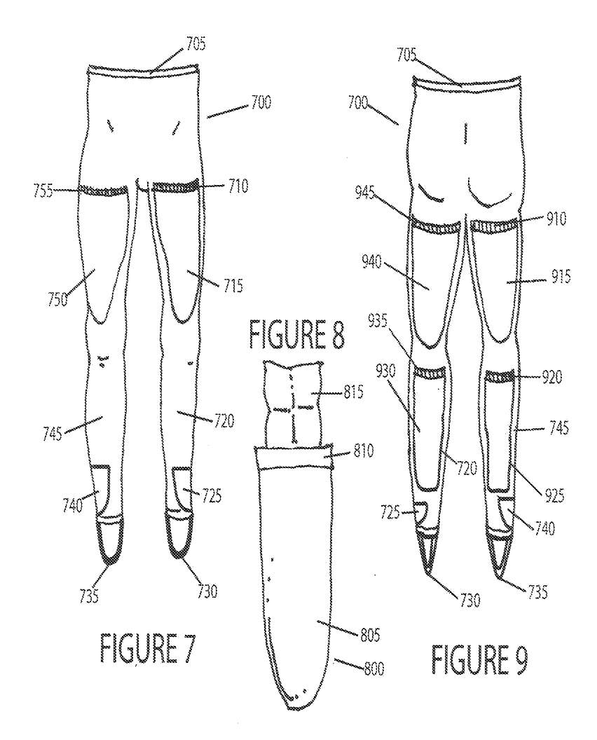

Referring now to FIG. 9, a rear view of the exemplary pants 700 from FIG. 7 having hamstring pockets 915, 940 according to principles of the first embodiment of the invention is shown. Additionally, calf pockets 925, 930 and optional ankle pockets 725, 740 are provided.

Each pocket 915, 925, 930, 940, includes an opening with an elastic band 910, 920, 935, 945 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

Pockets are preferably sized and positioned to target determined anatomical areas (muscles, tendons, bones, nerves and other tissue) of the wearer's body. By way of illustration and not limitation, for an adult's garment, a quadriceps pockets 715, 750 may be approximately 8″W×10″L and located on the front part of the middle upper thigh. Major muscle groups targeted by quadriceps pockets 715, 750 include, for example, Sartorius, Rectus Femoris, Vastus Medialis, and Vastus Lateralis. As another example (but without limitation), hamstring pockets 915, 940 for an adult's garment may be approximately 8″W×10″ L and located on the rear of the middle upper leg. Major muscle groups targeted by hamstring pockets 915, 940 include, for example, Semimembranosis and Biceps Femoris.

As yet another example (but without limitation), calf muscle pockets 925, 930 for an adult's garment may be approximately 8″L×6″W and located on the rear of the lower leg. Major muscle groups and (optionally) tendons targeted by calf muscle pockets 925, 930 include, for example, Gastrocnemius, Soleus and Achilles Tendons. If the Achilles Tendon is targeted, the calf muscle pockets 925, 930 may extend to the ankles.

The pockets identified above are examples, but not an exhaustive list, of pockets that may be included on pants in accordance with the principles of the various embodiments of the present invention. Such pants may include one or more of any of the aforementioned pockets, and/or pockets positioned to target other anatomical regions.

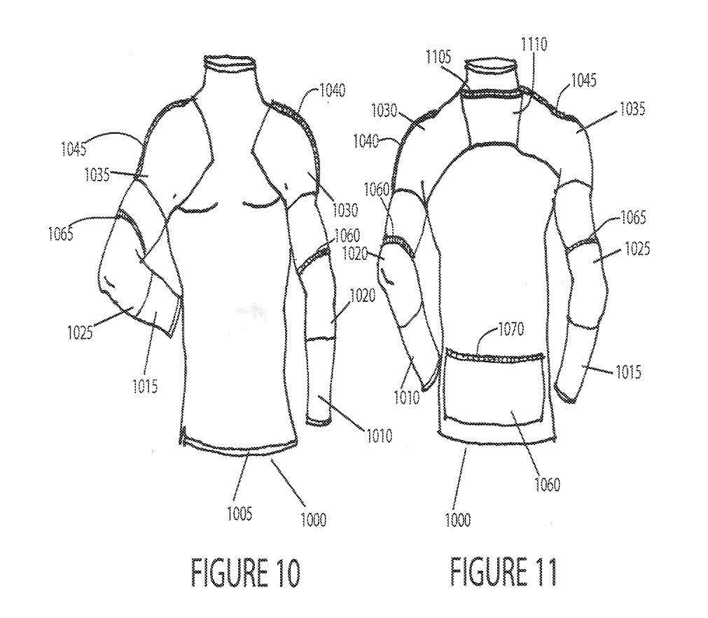

Referring now to FIG. 10, a front view of an exemplary form-fitting, stretchable, pull-over shirt 1000 having elbow 1020, 1025 and shoulder 1030, 1035 pockets according to principles of the first embodiment of the invention is shown. The shirt 1000 may be comprised of an elastic material as described above. An open waist with a waistband 1005, an open neck, a pair of sleeves 1010, 1015 and a wrist opening in each arm covering are provided. The shirt 1000 is preferably made to fit tightly over and conform to the contours or physique of the body of the wearer of the garment. Various panels of material may be stitched together in conventional manner to form the shirt. While the exemplary sleeves 1010, 1015 extend to about the wearer's wrists, shorter (or longer) sleeves may optionally be used.

Each of the elbow 1020, 1025 and shoulder 1030, 1035 pockets includes an opening with an elastic band 1040, 1060, 1045, 1065 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

Now referring now to FIG. 11, a rear view of the exemplary shirt 1000 having elbow 1020, 1025 and shoulder 1030, 1035 pockets, as well as a lower back (i.e., lumbar) pocket 1060, and a trapezius 1110 pocket, according to principles of the first embodiment of the invention is shown. The lower back pocket 1060 may optionally include an opening with an elastic band 1070 to facilitate closure. Likewise, the trapezius pocket 1110 may optionally include an opening with an elastic band 1105 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

The shirt pockets are preferably sized and positioned to target determined anatomical areas (muscles, tendons, bones, nerves and other tissue) of the wearer's body. By way of illustration and not limitation, for an adult's garment, shoulder pockets 1030, 1035 may be approximately 6″W×8″L and located on the topside of the upper sleeve. Major muscle groups targeted by shoulder pockets 1030, 1035 may include Trapezius, Deltoid, Biceps brachii, Brachialis, Brachial plexus, Brachioradialis, Coracobrachialis, Latissimus dorsi, Subclavius, Subscapularis, Supraspinatus, Teres major, Triceps brachii.

As with the pants pockets, the shirt pockets are adapted to hold thermal media 815 for treating targeted areas. Various thermal media now known in the pertinent art and hereafter developed may be utilized for delivering thermal therapy to a targeted area. Such media may include (without limitation) ice and heat packs and pads and hot water bottles. The principles of the invention apply to both cold thermal media and hot thermal media, both of which are intended to come within the scope hereof. Preferably, flexible media is used to enable the media to conform closely to the contours or physique of the body of the wearer of the garment. In a particular preferred embodiment, Techni Ice reusable dry ice packs/gel packs by Techni Ice Australia Pty Ltd are utilized for hot and cold therapies.

As another example (but without limitation), elbow pockets 1020, 1025 for an adult's garment may be approximately 6″W×6″ L and located on the outer elbow region of each sleeve. Major muscle groups targeted by elbow pockets 1020, 1025 may include, for example, portions of Anconeus (cubitalis rolani), Brachioradialis, Extensor carpi radialis brevis, Extensor carpi radialis longus, Extensor carpi ulnaris, Extensor digiti minimi, Extensor digitorum, Flexor carpi ulnaris, Biceps brachii, Brachialis, Brachioradialis, Flexor carpi radialis, Flexor carpi ulnaris, Palmaris longus, and Pronator teres.

As yet another example (but without limitation), a lower back (e.g., lumbar) pocket 1060 for an adult's garment may be approximately 12″L×6″W and located on the lower portion of the rear of the shirt 1000. Major muscle groups targeted by the lower back pocket 1060 may include, for example, portions of Quadratus Lumborum, Erector Spinae, Latissimus Dorsi, Trapezius, and External Oblique.

The pockets identified above are examples, but not an exhaustive list, of pockets that may be included on shirts in according to principles of the first embodiment of the inventions is shown. Such pants may include one or more of any of the aforementioned pockets, and/or pockets positioned to target other anatomical regions, such as the chest (pectoral), front waist (abdominal) and other areas. Additionally, in an embodiment with an extended neck (i.e., a turtleneck), a pocket may be provided in the front and/or rear of the neck region.

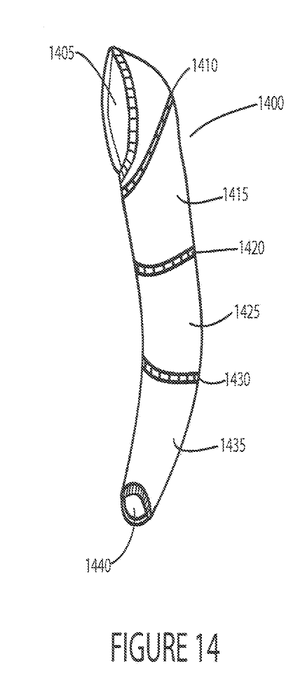

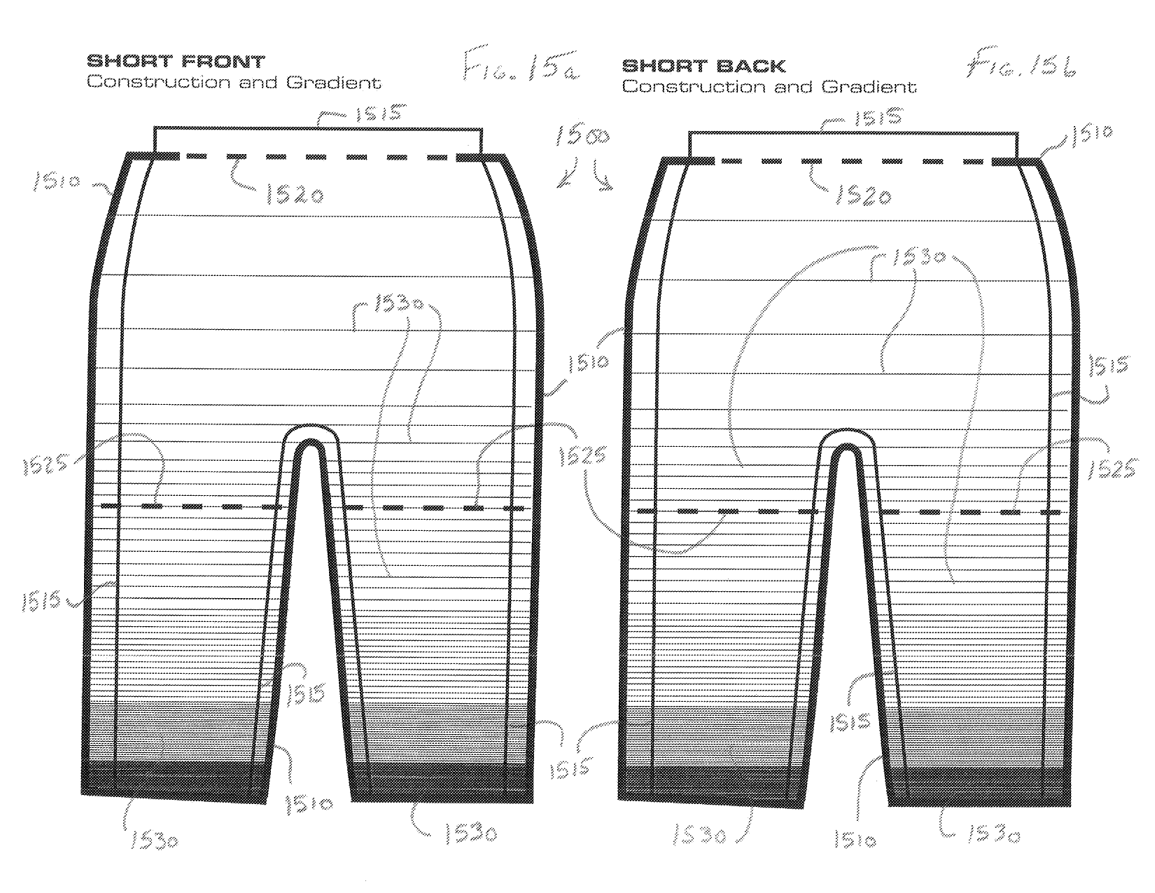

Referring now to FIG. 14, a front plan view of an exemplary form-fitting, stretchable, pull-on sleeve 1400 having an upper arm pocket 1415, elbow pocket 1425, and forearm pocket 1435. The pockets are sized and oriented to receive thermal media and maintain the media tightly against targeted muscle groups, such as biceps brachii and triceps brachii in the upper arm, the elbow joint, and forearm muscles including the flexors and extensors of the digits, a flexor of the elbow (brachioradialis), and pronators and supinators that turn the hand to face down or upwards, respectively. The sleeve 1400 may be comprised of an elastic material as described above. An open wrist with a wristband 1440 and an open shoulder 1405 with a shoulder band are provided. The sleeve 1400 is preferably made to fit tightly over and conform to the user's arm. Various panels of material may be stitched together in conventional manner to form the sleeve 1400. While the exemplary sleeve 1400 shown in FIG. 14 extends from the wearer's shoulder to about the wearer's wrists, shorter (or longer) sleeves may optionally be used within the spirit and scope of the invention.

Each of the upper arm pocket 1415, elbow pocket 1425, and forearm pocket 1435 includes an opening with an elastic band 1410, 1420, 1430 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

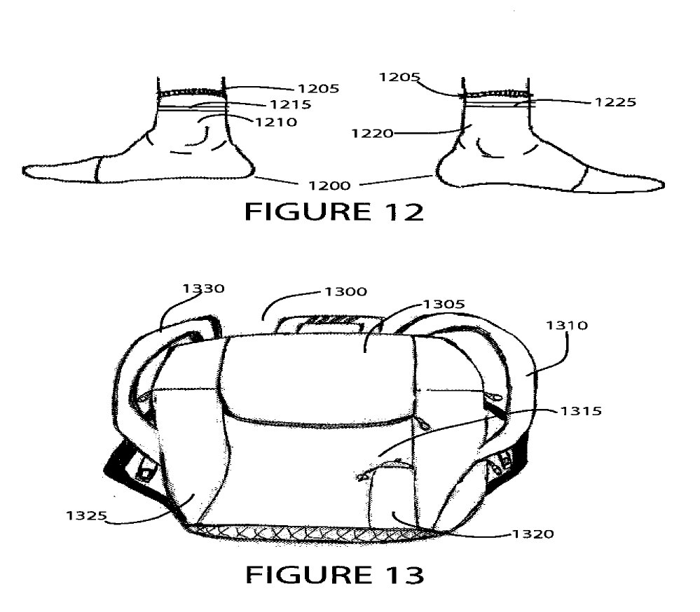

In yet another embodiment, a garment according to principles of the first embodiment of the invention comprises hosiery such as a sock 1200, as shown in FIG. 12, with one or more pockets 1210, 1220 in the area of the Achilles tendon. An opening with an elastic band 1205 is provided in a conventional manner. Each pocket 1210, 1220 may optionally include an opening with an elastic band 1215, 1225 to facilitate closure. Other forms of closure, such as hook and loop fasteners, snaps and buttons may be utilized in addition to or in lieu of elastic bands. Additionally, the closure may be omitted without departing from the scope of the invention, especially in a preferred feature of the first embodiment in which the pocket itself provides compression, which is typically sufficient to maintain the thermal pack in a compressive relationship against the underlying body part without risk of having the thermal pack move, much less come out of the pocket involuntarily or accidentally.

Yet another embodiment comprises one or more garments as described above, in accordance with principles of the invention, in a thermal kit. The kit includes a thermal container such as a duffel bag or backpack with a thermally insulated compartment for storing chilled/frozen thermal media (or heated thermal media) for an extended period of time, and another compartment (or the same compartment) for storing the garment. As shown in FIG. 13, an exemplary backpack 1300 is comprised of a container with a releasable closure 1305, and attached shoulder straps 1310, 1330. The closure 1305 provides access to one or more internal compartments, such as an insulated compartment for storing thermal media 1315 and mesh compartments 1325 for storing other items. Optionally, the backpack 1300 may also include a water bottle. The most common materials for such packs are canvas and nylon, either ripstop, ballistic or sack cloth fabrics. Thus, an athlete may bring the kit with him or her to a training session or an event. The heated or chilled thermal media will maintain an effective hot or cold temperature for several hours within the insulated compartment 1315 of the container. After training or completing the event, the athlete may immediately commence thermal therapy using the garment and thermal media, targeting specific muscle groups without the inconvenience and discomfort of an ice bath.

The invention provides several advantages. First, the invention is portable and facilitates thermal therapy immediately after strenuous activity, which enhances the restoration process. Another advantage is that fatigued and strained muscle groups and other anatomical areas may be targeted with thermal therapy by inserting thermal media into corresponding pockets, while other part of the body may remain at a comfortable ambient temperature. Yet another advantage is that the garment provides support and compression to targeted muscle groups, which enhance the recovery process.

In a second primary embodiment of the present invention, improved compression garments provide zoned and gradient compression and enable thermal therapy to be applied in numerous locations or on numerous body parts simultaneously and in a manner that enhances the healing and recovery process. Preferably, such compression garments providing zoned and gradient compression and include a dual layer of compressive seamless fabric that allows pockets to be defined therebetween at selective locations, accessible through slits or similar openings in the outer layer of the compressive fabric, for easily receiving and securely holding in place thermal media, such as ice or heat packs, that allows the application of targeted, compressive, and uniform thermal therapy to desired body part locations of the wearer of such a garment. Such garments can be used to apply targeted, compressive heat therapy to numerous body part locations during warm-ups, rehabilitation or physical therapy sessions, during exercise or actual sporting events. Similarly, these same garments can be used to apply targeted cold compressive therapy quickly and easily, immediately after exercise or shortly after an injury or other body trauma. The improved pocket design, which provides large pocket spaces between the dual compression garment layers, combined with the actual compression garments in which the fabric layers provide zoned, gradient, and transitional compression features, provides for an improved compression garment and one that more easily allows compressive thermal therapy to be applied uniformly to a wider range of muscle groups and other body parts, to expanded coverage of such muscle groups and other body parts—all of which are easily and quickly selectable by the wearer of the garment.

The improved therapeutic compression garments described hereinafter provide improved compression and thermal therapy benefits not heretofore available—particularly for individuals in the immediate period of time just after injury, trauma, or strenuous athletic activity. In addition, improved pocket designs and pocket locations used with such compression garments are described hereinafter that provide for more exact and enhanced thermal therapy at strategic muscle/joint/tendon/ligament locations and with improved and uniform coverage of desired muscle, joint, ligament, and tendon groups that are likely to need and benefit from immediate thermal therapy for warm-ups and stretching before athletic or rehabilitative activities or immediately after an injury, trauma, exercise, or rehab.

Preferably, the garments provide and are intended to provide or deliver gradient compression to all body parts underlying such garments and to apply additional, targeted or zoned compression to targeted body parts areas. The specific level of compression will vary by garment and by the intended use of such garment. The particular fabrics and weaves used to create zoned, compression garments are known to those skilled in the art. Further, it is possible and often desirable to include transition areas in which the level of compression gradually decrease as one moves away from a targeted or zoned compression area to a standard gradient compression area of the garment.

In another preferred embodiment, the design of the compression garments described herein takes advantage of the seamless technology offered by circular knitting machines to provide a garment that offers maximum comfort and flexibility, particularly when being used for warm-ups or during physical activity, while also providing maximum, uniform coverage and efficacy of compressive thermal therapy to desired areas of the wearer's body. Circular or seamless fabric construction does this by creating an equal surface pressure to keep either hot or cold pack (or sheets, as they may sometimes be called) not only in place but also under compression, and in a manner in which the thermal medium conforms to the contour or physique of the underlying body part being treated. Preferably, pockets for holding the thermal media are created and defined between dual fabric layers of the compression garment.

Preferably, the dual layers of fabric used to create such compression garments are made using a circular, multi-function knitting machine. Each of these garments is anatomically designed to create graduated or gradient compression—with highest compression further from the heart and lowest compression closer to the heart. Additionally, the compression is preferably zoned, meaning that support and compression provided by the garment increases and decreases based on its proximity to large muscle groups that are typically targeted for thermal therapy. This is preferably accomplished through the selection and use of certain weaves within the fabric. The balanced surface pressure that compression garments provide triggers improved blood circulation, which delivers more oxygen to working muscles. Better circulation also enables the body to eliminate lactic acid and other metabolic wastes that can cause muscle fatigue. The result is an anatomically specific graduated compression garment that fits like a second skin. Yet further, through the use of transitional areas between zoned compression areas that provide the highest level of compression and non-zoned areas that provide the least level of compression, the garments described herein provide significant flexibility and range of movement and minimize any restriction to the wearer's movement while wearing such compression garments—whether or not thermal therapy is being applied at the time.

In a preferred embodiment, the fabric used to create the compression garments described herein are made from high performance, 4-way stretch yarns. This fabric design improves comfort, mobility, and moisture control in both the inner and outer layer, when the compression garment includes dual layers. Additionally, such fabric allows for an efficient transfer of the thermal therapy to the desired body part of the wearer. In some embodiments, the thickness of the two layers of the garment are substantially the same. In other embodiments, the inner layer of the garment (i.e., the one closest to the skin of the wearer is thinner than the outer layer of fabric to improve the thermal transfer between the wearer's body and any thermal packs inserted in pockets between the dual layers of fabric.

In one embodiment, both layers of the garment have gradient and zoned compression properties. In other embodiments, the inner layer of the garment is substantially, uniformally compressive, but the outer layer of the garment provides the gradient and zoned and transitional compressive aspects of the garment as a whole.

The pocket architecture of the improved compression garment described herein are preferably achieved through the ability of the circular knitting and 4 way stretch fabric to reduce the number of seams needed to create what is referred to as the pocket. Preferably, such pockets are formed and created between the dual layers of compression fabric used to create the compression garment. The outer layer of the garment provides the necessary zoned and gradient compression, which is sufficient to hold ice or heat packs (or similar thermal therapy media) in place and in compression, while still allowing for maximum mobility and comfort for the wearer of the compression garment. Although the fabric used to make the compression garments is described as circular or seamless fabric construction, the compression garments described herein use seams or hems to attached the two layers of the garment together at desired or strategic locations. Such seams or hems are used to create and reinforce the pocket openings, are used to attached the two layers of the fabric together to define the outer confines of different pockets contained between the two layers of garment fabric, and to provide other reinforcement horizontally or vertically along the document. Preferably, such seams or hems are designed and use threads that enable the seam or hem to stretch with the surrounding garment fabric and without inhibiting the elastic, flexibility, stretchability and recoverability of the overall compression garment. Using the space between the dual layers of compression garment fabric to define and place the pockets for holding the thermal media enables pocket openings to be placed in a wide range of locations, depending upon the type of garment involved and the particular uses for which that garment will be used. Such pocket design also allows for the pockets to be larger than traditional pockets, whether they be sewn behind the primary or only layer of fabric used in conventional garments or whether they are attached to the outside surface of a traditional garment. Thus, the present compression garments provide for maximum and targeted coverage for use of thermal therapy. In other words, thermal therapy is not limited to small pocket areas, but to almost any space, and, likewise, to any underlying body part of the wearer that needs application of thermal therapy.

Specific compression garments used in conjunction with this second preferred embodiment of the present invention include, but are not limited to: shorts, which are preferably intended to cover the quads, hamstrings, it band, hip flexors, and lower lumbar region; knickers, which are similar to shorts but also preferably include coverage of the patella and surrounding tendons; tights, which are similar to knickers but also preferably include coverage of the shin and calf; long pants, which are similar to tights but which go down to or over the ankles; shin and calf sleeves, which are preferably intended to cover the shins and calves, respectively; knee sleeves, which are preferably intended to cover the front, radial, and rear portions of the knee; elbow sleeves; wrist sleeves; wrist/hand gloves or glove/sleeve combinations; forearm sleeves, full arm sleeves, which are preferably intended to cover the wrist, forearm, and elbow (front and rear); short sleeve shirts, which are preferably intended to cover the shoulder head (front and rear), scalpula, L1 and L2, L4, and L5 of the vertebrae; long sleeve shirts, which are preferably intended to cover everything that the short sleeve shirt covers plus includes the same coverage provided by the arm sleeves, plus coverage of the biceps and triceps muscle groups; long socks and/or ankle sleeves, which are preferably intended to cover the ankle (inside and out), the top of the foot, and in some embodiments the areas affected by plantar fasciitis; full body tights, which are intended to cover the same areas as long or short sleeve shirts in combination with one of shorts, knickers, tights, and long pants. Compression garments may also include girdles or similar body wraps that are designed only to cover some or all of the torso part of the body, but none of the appendages as well as any type of head garments, such as a ski mask with or without coverage of any facial areas.

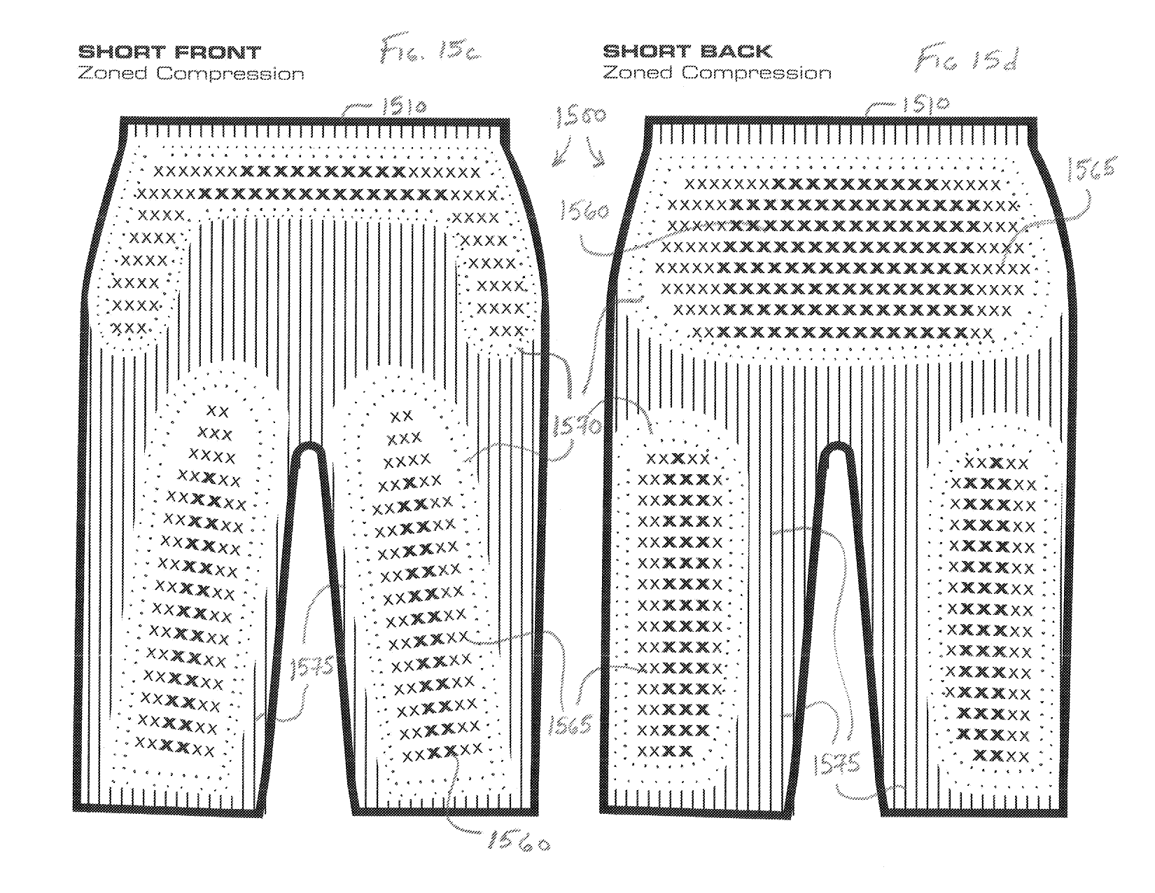

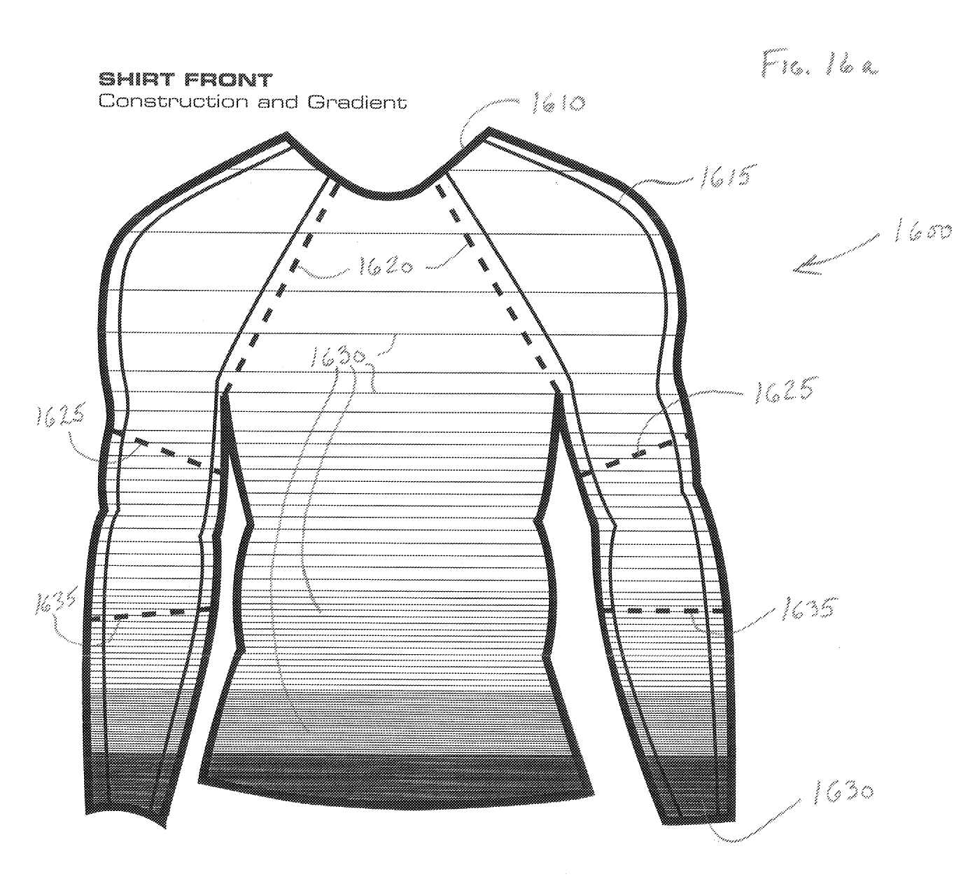

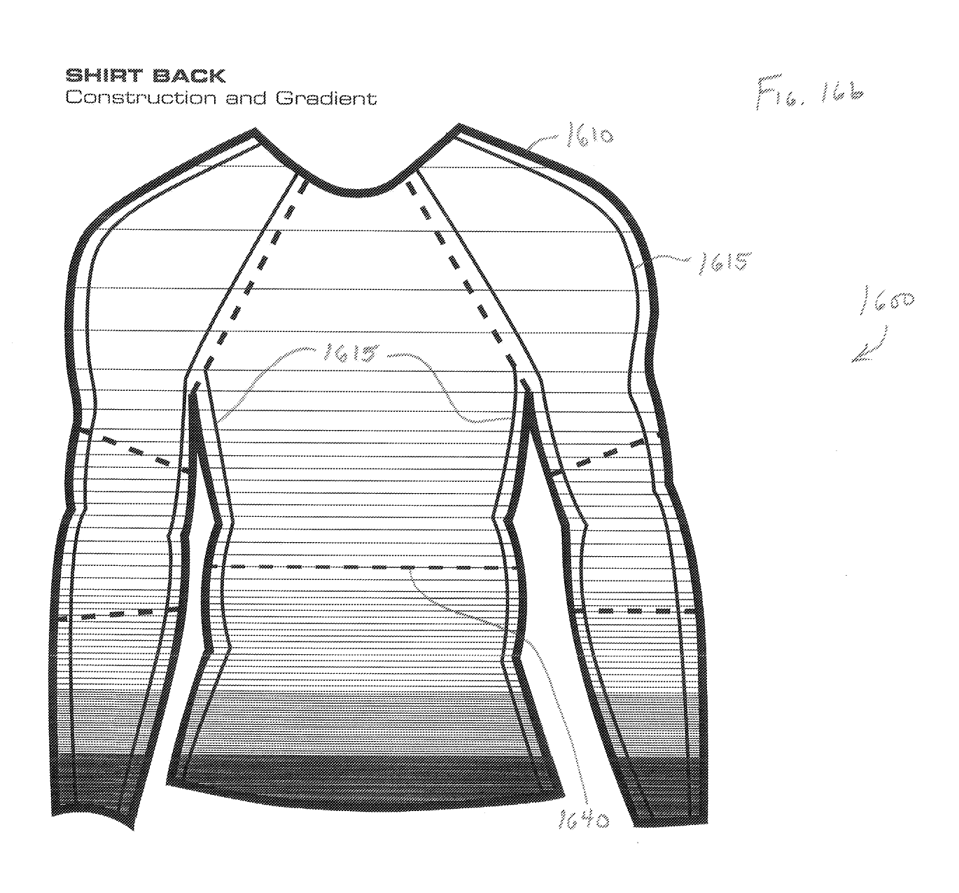

Turning now to FIGS. 15 through 18, a more detailed explanation and illustration of gradient compression and zoned compression is provided. Further, use of the dual layers of compression fabric and location of pocket openings, slits, or seams for several different exemplary garments are also illustrated.

Turning first to FIGS. 15a-15d, an exemplary pair of compression shorts 1500 according to this second embodiment of the present invention is shown. FIGS. 15a and 15b show a plan view of the front and back sides, respectively, of the compression shorts 1500. Likewise, FIGS. 15c and 15d show a plan view of the front and back sides, respectively, of the compression shorts 1500.

FIGS. 15a and 15b illustrate, more specifically, the use of the dual or double layer of fabric used to create the compression shorts 1500 and that are used to define a space therebetween to create one or more pockets to hold one or more thermal media therein. The outer, exterior, or second layer of fabric 1510 is a graduated and zoned compression fabric, as defined and described previously. The inner, interior, or first layer of fabric 1515 is either a simple compression garment fabric or, alternatively, a graduated and zoned compression fabric similar to the outer, exterior, or second layer of fabric 1510. The outer, exterior, or second layer is of fabric 1510 is the layer of fabric of the garment that is typically visible to others. The inner, interior, or first layer of fabric 1515 is typically the layer of fabric that is adjacent to the skin of the wearer of the garment—unless the wearer is wearing an undergarment, wrap, or bandage under such layer 1515 on all or some part of his body underneath the garment. Preferably, however, there is direct contact between the inner, interior, or first layer of fabric 1515 and the skin of the wearer of the garment to enhance the effectiveness of any thermal therapy applied while wearing the garment. Because both layers of fabric are compressive, but elastic and stretchable, any thermal medium can be easily inserted by the wearer of the garment (or a third party) between the two fabric layers and can be strategically placed or targeted adjacent to or over a specific body part location, as desired by the wearer of the garment. Further and preferably, the compressive aspects of the outer layer 1515 of the garment ensure that, once placed, not only will such thermal medium remain held in the desired location, but will be held compressively against the desired body part of the wearer, with only the inner layer of fabric 1510 interposed between the thermal pack or sheet and the underlying body part of the wearer. In some embodiments, the thickness and thermal transfer characteristics of both layers of the garment are substantially the same. In other embodiments, the inner, interior, or first layer of fabric 1515 is thinner and/or has improved thermal transfer characteristics to enhance the effect of the inserted thermal medium inserted in the pocket area

The pocket openings are shown in FIGS. 15a and 15b as dashed lines 1520 and 1525. The fabric layers above the pocket openings 1520, 1525 are typically stitched, hemmed, or otherwise adhered together. The end points of the pocket openings 1520, 1525 are also preferably reinforced with stitching or extend to existing vertical stitching of the garment to minimize the risk of tearing or ripping. The pocket opening is, at a minimum, at least wide enough to allow a thermal pack or sheet to be inserted therein and, in some embodiments, is wide enough to allow the wearer to insert his hand therein to assist in placement of the thermal medium. It should be noted, however, that the pocket opening does not have to be wide enough for the wearer's hand because, once inserted, it is relatively easy for the wearer to stretch the outer layer of fabric 1515 away from his body and maneuver the thermal medium into its desired location by moving and sliding the thermal medium through or with the outer layer of fabric 1510 until it is in its desired location within the pocket. In some embodiments, the pocket opening extends fully across the upper layer 1510 of fabric and stops at each end of the opening at the vertical stitching or seam used to adhere the two layers of fabric 1510, 1515 together. The actual pocket (not shown) is defined or created by the space below the pocket openings 1520, 1525 and between the two layers of fabric 1510, 1515. Preferably, the outer edges of each pocket are defined by seams sewn between the two layers of fabric, usually and preferably in locations that minimize any restrictions on movement, stretching or flexibility of the overall garment and also at locations above any pocket openings that are placed lower on the same garment. Thus, for the compression shorts 1500 of FIGS. 15a and 15b, the pocket accessed through opening 1520 would close off, preferably, above pocket opening 1525. As will be appreciated by those skilled in the art, however, in alternative embodiments (not shown), the entire space (or substantial portion of space) between the two layers of the garment could define a single, large pocket area having one or more pocket openings into the same pocket.

Finally, still referring to FIGS. 15a and 15b, the horizontal lines 1530 are not an actual part of the garment but are used graphically to illustrate the graduated or increasing gradient pressure or compression applied generally by the garment fabric as a whole. The further such lines 1530 are spaced from each other, the lower the relative compression in that part of the garment. The closer the lines 1530 are to each other, the higher the relative compression. As has been stated previously, it is desirable for most compression garments to have less gradient compression on parts of the body closer to the heart, with increasing gradient compression applied by portions of the garment further from the heart.

FIGS. 15c and 15d illustrate, more specifically, where and how zoned compression and transitional compression areas are used and designed into the outer layer 1510 of fabric of a garment to target compression on key muscle groups or other body parts underlying the specific garment, the exemplary compression shorts 1500, in this case. The dark or bolded X symbols 1560 are indicative, graphically, of areas of greatest compression applied in that “zone” or area of the garment. The lighter colored X symbols 1565 are indicative, graphically, of areas of compression that are a step down from the areas designated by the bolded X symbols. The dotted symbols 1570 indicate transition compression areas that are yet a further step down in compression level as compared to the lighter colored X symbols. The vertical lines 1575 are indicative of the minimum compression level generally applied and as compared to the higher zoned compression areas 1560, 1565, 1570. Although not shown in FIGS. 15c and 15d, it should be understood that the zoned compression levels illustrated in FIGS. 15c and 15d coexist and overlap with the gradient pressure levels illustrated by lines 1530 in FIGS. 15a and 15b. It will be appreciated by those skilled in the art that the zoned and gradient compression can be configured and designed into the layers of the garment based on selection and combination of fabric and weave chosen by the manufacturer of the garment layers.

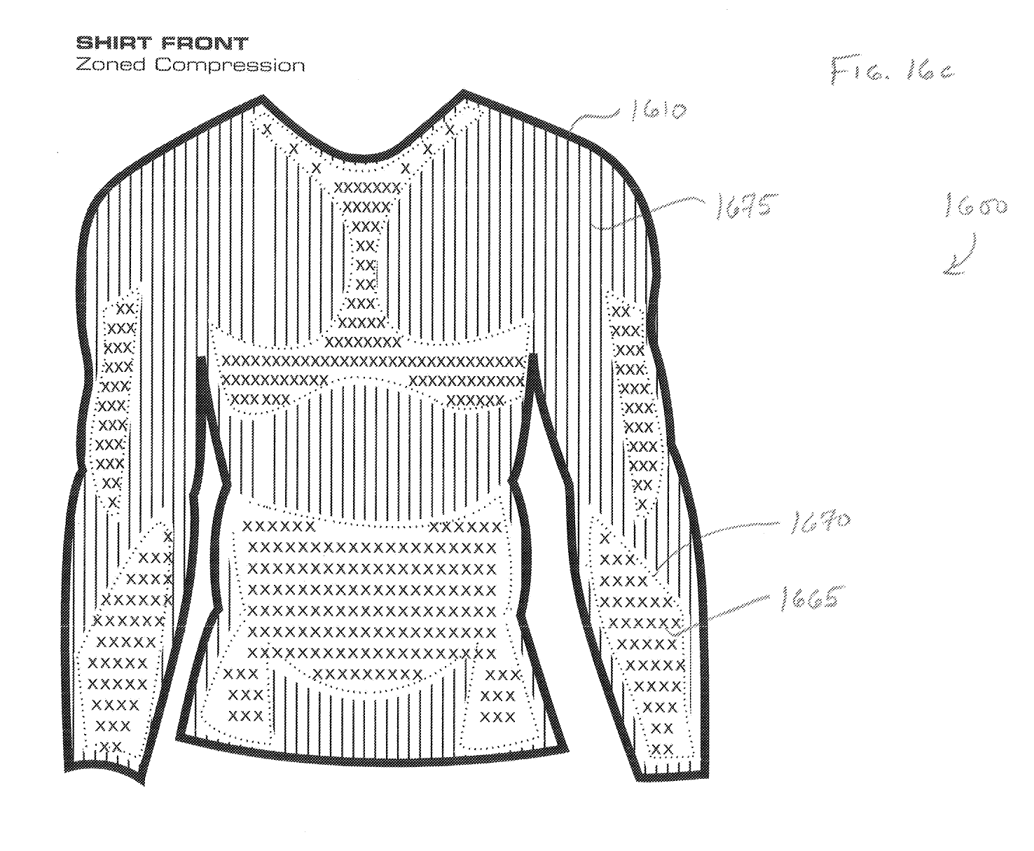

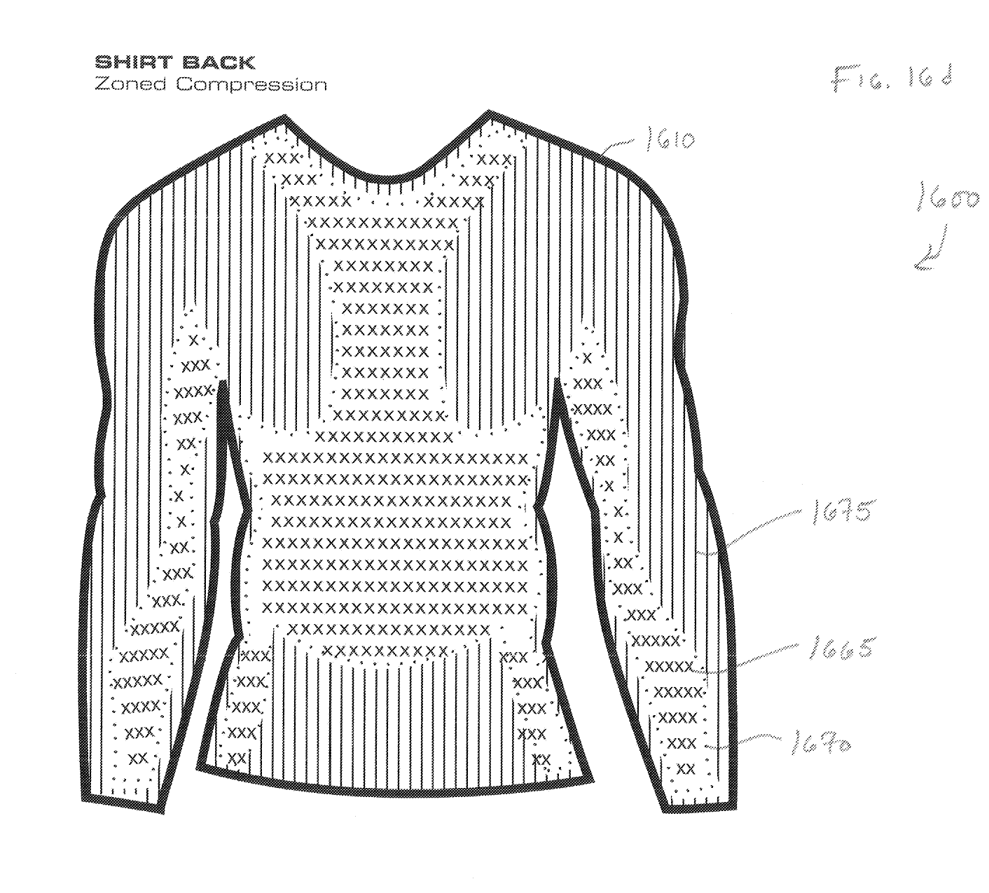

Turning now to FIGS. 16a-16d, an exemplary long sleeve compression shirt 1600 according to this second embodiment of the present invention is shown. FIGS. 16a and 16b show a plan view of the front and back sides, respectively, of the long sleeve compression shirt 1600. Likewise, FIGS. 16c and 16d show a plan view of the front and back sides, respectively, of the long sleeve compression shirt 1600.

FIGS. 16a and 16b illustrate, more specifically, the use of the dual or double layer of fabric used to create the long sleeve compression shirt 1600 and that are used to define a space therebetween to create one or more pockets to hold one or more thermal media therein. The outer, exterior, or second layer of fabric 1610 is a graduated and zoned compression fabric, as defined and described previously. The inner, interior, or first layer of fabric 1615 is either a simple, uniform compression fabric or, alternatively, a graduated and zoned compression fabric similar to the outer, exterior, or second layer of fabric 1610. The outer, exterior, or second layer of fabric 1610 is the layer of fabric of the garment that is typically visible to others. The inner, interior, or first layer of fabric 1615 is typically the layer of fabric that is adjacent to the skin of the wearer of the garment—unless the wearer is wearing an undergarment, wrap, or bandage under such layer 1615 on all or some part of his body underneath the garment. Preferably, however, there is direct contact between the inner, interior, or first layer of fabric 1615 and the skin of the wearer of the garment to enhance the effectiveness of any thermal therapy applied while wearing the garment. Because both layers of fabric are compressive, but elastic and stretchable, any thermal medium can be easily inserted by the wearer of the garment (or a third party) between the two fabric layers and can be strategically placed or targeted adjacent to or over a specific body part location, as desired by the wearer of the garment. Further and preferably, the compressive aspects of the outer layer 1615 of the garment ensure that, once placed, not only will such thermal medium remain held in the desired location, but will be held compressively against the desired body part of the wearer, with only the inner layer of fabric 1610 interposed between the thermal pack or sheet and the underlying body part of the wearer. In some embodiments, the thickness and thermal transfer characteristics of both layers of the garment are substantially the same. In other embodiments, the inner, interior, or first layer of fabric 1615 is thinner and/or has improved thermal transfer characteristics to enhance the effect of the inserted thermal medium inserted in the pocket area

The pocket openings are shown in FIGS. 16a and 16b as dashed lines 1620, 1625, 1635, and 1640. Pocket openings 1620 provide access to the shoulder area pockets of the wearer of the long sleeve compression shirt 1600. Pocket openings 1625 provide access to the elbow area pockets of the wearer of the long sleeve compression shirt 1600. Pocket openings 1635 provide access to the forearm and/or wrist area pockets of the wearer of the long sleeve compression shirt 1600. Pocket opening 1640, shown only in FIG. 16b, provides access to the lower back pockets of the wearer of the long sleeve compression shirt 1600. In this particular long sleeve shirt example, there are no pockets provided for the stomach area of the wearer. This illustrates the general principal that the number and type of pockets chosen for any particular garment is variable and is a mere matter of choice by the garment designer or manufacturer. Further, it should be understood that the specific locations chosen for the pocket openings is also variable and can be placed at any location that is convenient, accessible. Aesthetics and appearance considerations of the garment are also a basis for selecting where to place pocket openings. Further, the intended use of the garment will also impact where pockets may be located and where the pocket openings are placed in association therewith.

As with the compression shorts 1500 of FIGS. 15a-15d, the fabric layers above the various pocket openings 1620, 1625, 1635, and 1640 of the long sleeve compression shirt 1600 are typically stitched, hemmed, or otherwise adhered together. The end points of the various pocket openings 1620, 1625, 1635, and 1640 are also preferably reinforced with stitching or extend to existing vertical stitching of the garment to minimize the risk of tearing or ripping. Generally, each pocket opening is, at a minimum, at least wide enough to allow a thermal pack or sheet to be inserted therein and, in some embodiments, is wide enough to allow the wearer to insert his hand therein to assist in placement of the thermal medium. It should be noted, however, that the pocket opening does not have to be wide enough for the wearer's hand because, once inserted, it is relatively easy for the wearer to stretch the outer layer of fabric 1615 away from his body and maneuver the thermal medium into its desired location by moving and sliding the thermal medium through or with the outer layer of fabric 1610 until it is in its desired location within the pocket. In some embodiments, the pocket opening extends fully across the upper layer 1610 of fabric until each end of the opening reaches a respective vertical stitching or seam used to adhere the two layers of fabric 1610, 1615 together. The actual pockets (not shown) are defined or created by the space below the pocket openings 1620, 1625, 1635, and 1640 and between the two layers of fabric 1610, 1615. Preferably, the outer edges of each pocket are defined by seams sewn between the two layers of fabric, usually and preferably in locations that minimize any restrictions on movement, stretching or flexibility of the overall garment and also at locations above any pocket openings that are placed lower on the same garment. Thus, for the long sleeve compression shirt 1600 of FIGS. 16a and 16b, the pocket accessed through opening 1620 would close off, preferably, above pocket opening 1625. Likewise for the pocket accessed through opening 1625, which would close off, preferably, above pocket opening 1635. The pocket accessed through opening 1635 would close off, preferably, at or near the hem attaching the two layers of fabric at the end of each sleeve. Similarly, the pocket accessed through opening 1640 would close off, preferably, at or near the hem attaching the two layers of fabric at the bottom edge of the shirt 1600. As will be appreciated by those skilled in the art, however, in alternative embodiments (not shown), the entire space (or substantial portion of space) between the two layers of the garment could define a single, large pocket area having one or more pocket openings into the same pocket.

Finally, still referring to FIGS. 16a and 16b, the horizontal lines 1630 are not an actual part of the garment but are used graphically to illustrate the graduated or increasing gradient pressure or compression applied generally by the garment fabric as a whole. The further such lines 1630 are spaced from each other, the lower the relative compression in that part of the garment. The closer the lines 1630 are to each other, the higher the relative compression. As has been stated previously, it is desirable for most compression garments to have less gradient compression on parts of the body closer to the heart, with increasing gradient compression applied by portions of the garment further from the heart.

FIGS. 16c and 16d illustrate, more specifically, where and how zoned compression and transitional compression areas are used and designed into the outer layer 1610 of fabric of a garment to target compression on key muscle groups or other body parts underlying the specific garment, the exemplary long sleeve compression shirt 1600, in this case. With the shirt, there are no dark or bolded X symbols shown, which would be indicative, graphically, of areas of greatest compression applied in that “zone” or area of the garment. The lighter colored X symbols 1665 are indicative, graphically, of areas of compression that are a step down from the areas designated by the bolded X symbols and indicate the highest level of zoned compression used for the long sleeve compression shirt 1600. Thus, it should be apparent that the highest zoned compression of the exemplary shirt 1600 is not as compressive as the highest zoned compression of the exemplary shorts 1500. The dotted symbols 1670 indicate transition compression areas that are yet a further step down in compression level as compared to the lighter colored X symbols. The vertical lines 1675 are indicative of the minimum compression level generally applied and as compared to the higher zoned compression areas 1665, 1670. Although not shown in FIGS. 16c and 16d, it should be understood that the zoned compression levels illustrated in FIGS. 16c and 16d coexist and overlap with the gradient pressure levels illustrated by lines 1630 in FIGS. 16a and 16b. It will be appreciated by those skilled in the art that the zoned and gradient compression can be configured and designed into the layers of the garment based on selection and combination of fabric and weave chosen by the manufacturer of the garment layers.

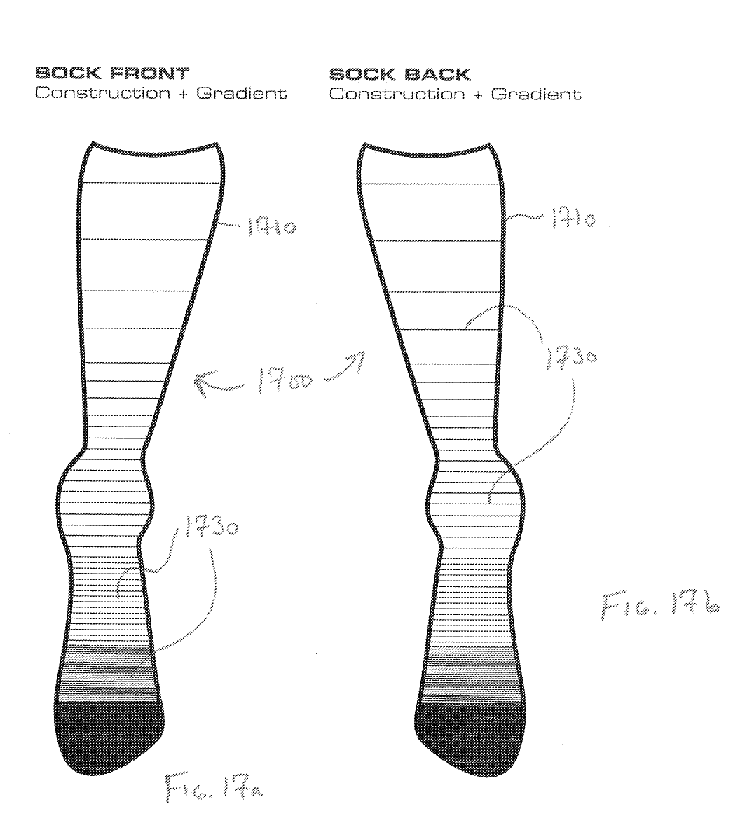

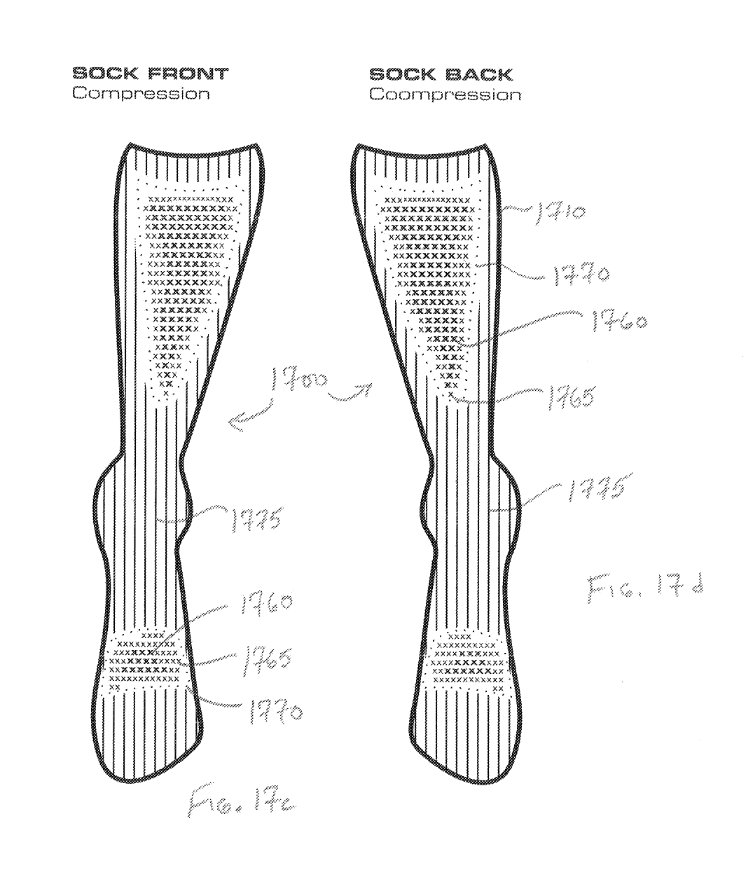

Turning now to FIGS. 17a-17d, an exemplary compression sock 1700 according to this second embodiment of the present invention is shown. FIGS. 17a and 17b show a plan view of the front and back sides, respectively, of the exemplary compression sock 1700. Likewise, FIGS. 17c and 17d show a plan view of the front and back sides, respectively, of the exemplary compression sock 1700.

FIGS. 17a and 17b illustrate, more specifically, the use of only a single layer 1710 of fabric used to create the exemplary compression sock 1700. Although not shown, an inner layer of fabric could be used along the entire length of the sock 1700, or could be selectively used to create an ankle and/or a calf pocket. The outer, exterior, or second layer of fabric 1710 is a graduated and zoned compression fabric, as defined and described previously. The inner, interior, or first layer of fabric, if used, would either be a simple, uniform compression fabric or, alternatively, a graduated and zoned compression fabric similar to the outer, exterior, or second layer of fabric 1710. Because an inner layer of fabric is not illustrated, there are no pocket openings illustrated. As stated previously, however, the location and choice of where to include pockets and corresponding openings are variable and are a mere matter of choice by the garment designer or manufacturer. Aesthetics and appearance considerations of the garment are also a basis for selecting where to place pocket openings. Further, the intended use of the garment will also impact where pockets may be located and where the pocket openings are placed in association therewith.

Finally, still referring to FIGS. 17a and 17b, the horizontal lines 1730 are not an actual part of the garment but are used graphically to illustrate the graduated or increasing gradient pressure or compression applied generally by the garment fabric as a whole. FIGS. 17c and 17d illustrate, more specifically, where and how zoned compression and transitional compression areas are used and designed into the outer layer 1710 of fabric of the garment to target compression on key muscle groups or other body parts underlying the specific garment, the exemplary compression sock 1700, in this case. The dark or bolded X symbols 1760 are indicative, graphically, of areas of greatest compression applied in that “zone” or area of the garment. The lighter colored X symbols 1765 are indicative, graphically, of areas of compression that are a step down from the areas designated by the bolded X symbols 1760. The dotted symbols 1770 indicate transition compression areas that are yet a further step down in compression level as compared to the lighter colored X symbols 1765. The vertical lines 1775 are indicative of the minimum compression level generally applied and as compared to the higher zoned compression areas 1760, 1765, and 1770. Although not shown in FIGS. 17c and 17d, it should be understood that the zoned compression levels illustrated in FIGS. 17c and 17d coexist and overlap with the gradient pressure levels illustrated by lines 1730 in FIGS. 17a and 17b. It will be appreciated by those skilled in the art that the zoned and gradient compression can be configured and designed into the layers of the garment based on selection and combination of fabric and weave chosen by the manufacturer of the garment layers.

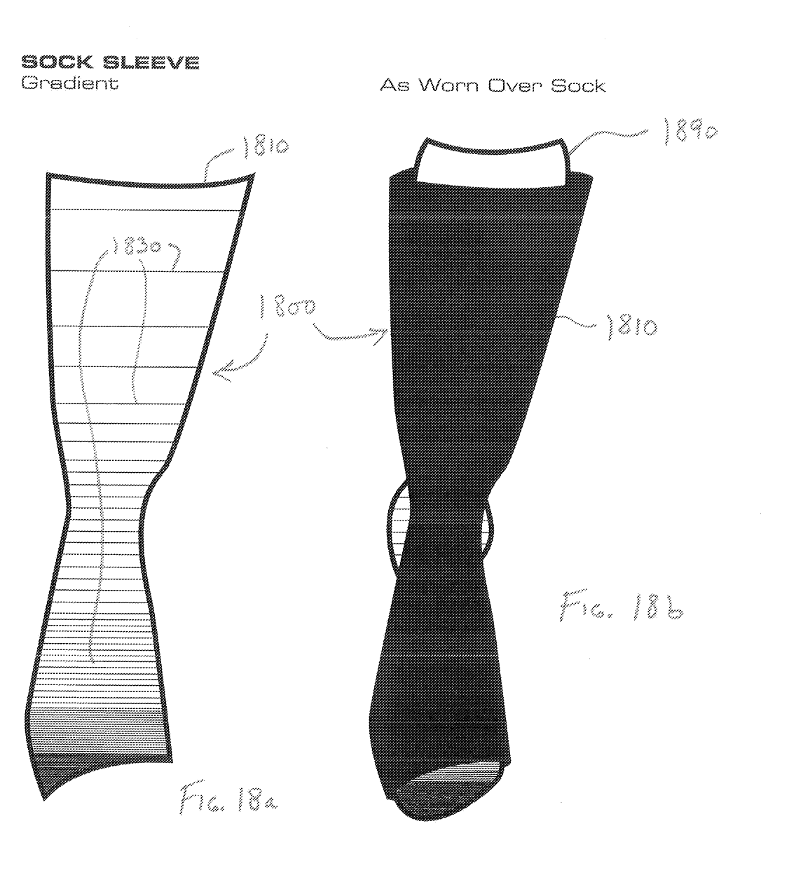

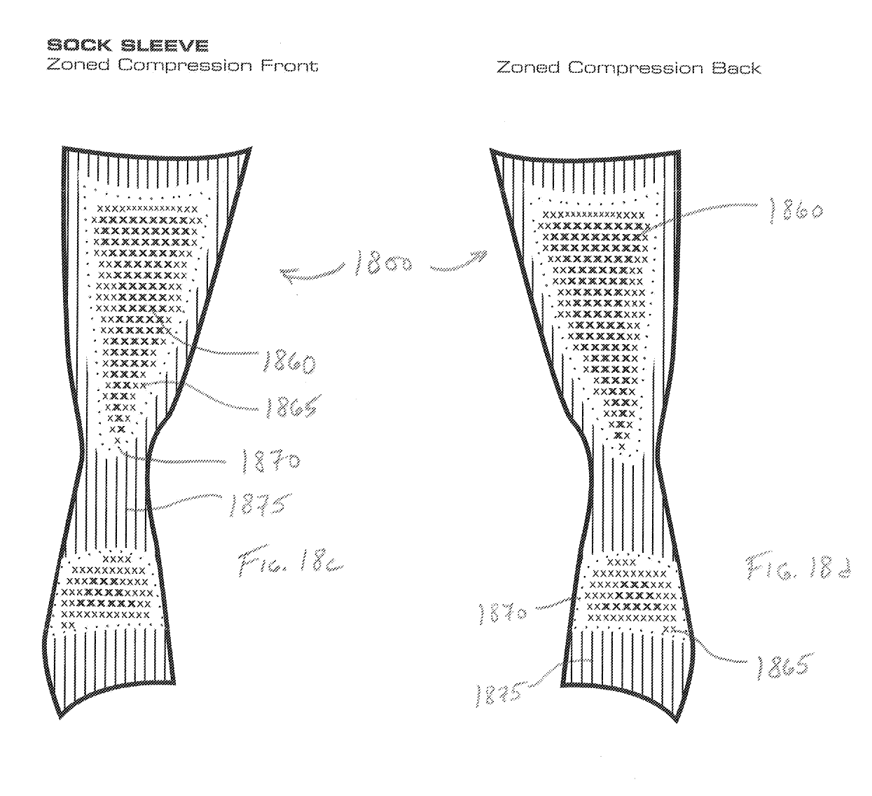

Turning now to FIGS. 18a-18d, an exemplary compression sock sleeve 1800 according to this second embodiment of the present invention is shown. FIGS. 18a and 18b show a plan view of the front and back sides, respectively, of the exemplary compression sock sleeve 1800. Likewise, FIGS. 18c and 18d show a plan view of the front and back sides, respectively, of the exemplary compression sock sleeve 1800.

FIGS. 18a and 18b illustrate, more specifically, the use of only a single layer 1810 of fabric used to create the exemplary compression sock sleeve 1800. Although not shown, an inner layer of fabric could be used along the entire length of the sock sleeve 1800, or could be selectively used to create an ankle and/or a calf pocket. The outer, exterior, or second layer of fabric 1810 is a graduated and zoned compression fabric, as defined and described previously. The inner, interior, or first layer of, fabric, if used, would either be a simple, uniform compression fabric or, alternatively, a graduated and zoned compression fabric similar to the outer, exterior, or second layer of fabric 1810. Because an inner layer of fabric is not illustrated, there are no pocket openings illustrated. As stated previously, however, the location and choice of where to include pockets and corresponding openings are variable and are a mere matter of choice by the garment designer or manufacturer. Aesthetics and appearance considerations of the garment are also a basis for selecting where to place pocket openings. Further, the intended use of the garment will also impact where pockets may be located and where the pocket openings are placed in association therewith.

FIG. 18b illustrates that the sock sleeve 1800 has a cut out for both the heel and the toe of the wearer. The actual sock 1890 being worn beneath the sock sleeve is shown.

Finally, still referring to FIGS. 18a and 18b, the horizontal lines 1830 are not an actual part of the garment but are used graphically to illustrate the graduated or increasing gradient pressure or compression applied generally by the garment fabric as a whole. FIGS. 18c and 18d illustrate, more specifically, where and how zoned compression and transitional compression areas are used and designed into the outer layer 1810 of fabric of the garment to target compression on key muscle groups or other body parts underlying the specific garment, the exemplary compression sock sleeve 1800, in this case. The dark or bolded X symbols 1860 are indicative, graphically, of areas of greatest compression applied in that “zone” or area of the garment. The lighter colored X symbols 1865 are indicative, graphically, of areas of compression that are a step down from the areas designated by the bolded X symbols 1860. The dotted symbols 1870 indicate transition compression areas that are yet a further step down in compression level as compared to the lighter colored X symbols 1865. The vertical lines 1875 are indicative of the minimum compression level generally applied and as compared to the higher zoned compression areas 1860, 1865, and 1870. Although not shown in FIGS. 18c and 18d, it should be understood that the zoned compression levels illustrated in FIGS. 18c and 18d coexist and overlap with the gradient pressure levels illustrated by lines 1830 in FIGS. 18a and 18b. It will be appreciated by those skilled in the art that the zoned and gradient compression can be configured and designed into the layers of the garment based on selection and combination of fabric and weave chosen by the manufacturer of the garment layers.

In view of the foregoing detailed description of preferred embodiments of the present invention, it readily will be understood by those persons skilled in the art that the present invention is susceptible to broad utility and application. While various aspects have been described in the context of aspects, features, and methodologies of the present invention will be readily discernable therefrom. Many embodiments and adaptations of the present invention other than those herein described, as well as many variations, modifications, and equivalent arrangements and methodologies, will be apparent from or reasonably suggested by the present invention and the foregoing description thereof, without departing from the substance or scope of the present invention. Furthermore, any sequence(s) and/or temporal order of steps of various processes described and claimed herein are those considered to be the best mode contemplated for carrying out the present invention. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in various different sequences and orders, while still falling within the scope of the present inventions. In addition, some steps may be carried out simultaneously. Accordingly, while the present invention has been described herein in detail in relation to preferred embodiments, it is to be understood that this disclosure is only illustrative and exemplary of the present invention and is made merely for purposes of providing a full and enabling disclosure of the invention. The foregoing disclosure is not intended nor is to be construed to limit the present invention or otherwise to exclude any such other embodiments, adaptations, variations, modifications and equivalent arrangements, the present invention being limited only by the claims appended hereto and the equivalents thereof.

We claim:

1. A therapeutic compression garment, comprising a dual layer of fabric including an outer layer and an inner layer, wherein the outer layer and inner layer are hemmed together along their respective edges to define the garment's shape and type, wherein the inner layer is comprised of compression fabric and wherein the outer layer is comprised of gradient compression fabric and wherein the outer layer further includes at least one area of zoned compression, wherein at least one pocket is defined between said outer and said inner layers of fabric and is bounded between said outer and said inner layers of fabric by one or more hems connecting the outer layer and inner layer of fabric together, wherein said pocket is positioned under said at least one area of zoned compression and is accessible through a slit formed in said outer layer of fabric, wherein the at least one pocket is strategically positioned within the garment at a location overlying a respective body part of an intended wearer of the garment, and wherein the at least one pocket is sized and shaped to receive at least one insertable thermal medium therein, whereby said at least one insertable thermal medium applies compressive thermal therapy to the respective underlying body part of the wearer as a function of the compression applied by the inner layer of fabric and of the combined gradient compression and zoned compression of the outer layer of the fabric.

2. The therapeutic compression garment of claim 1 wherein the dual layer of fabric stretches to fit the contours of the body of the wearer of the garment.

3. The therapeutic compression garment of claim 1 wherein said respective underlying body part of the wearer includes one or more of a muscle group, skin tissue, a bone, a joint, a tendon, a ligament, and nerves.

4. The therapeutic compression garment of claim 1 wherein the insertable thermal medium is flexible and conforms to the contours of the respective body part of the wearer when inserted into the at least one pocket.

5. The therapeutic compression garment of claim 1 wherein the compression garment's type and shape includes one of: a pair of shorts, knickers, tights, long pants, a shin and calf sleeve, a knee sleeve, an elbow sleeve, a wrist sleeve; a wrist/hand glove, a glove and sleeve combination, a forearm sleeve, a full arm sleeve, a short sleeve shirt, a long sleeve shirt, long socks, an ankle sleeve, a full body tights, a girdle, a body wrap, and a head covering.
